(19) United States
(12) Patent Application Publication
(10) Pub. No.: US 2024/0279142 A1
(43) Pub. Date: Aug. 22, 2024

(54) METHOD FOR PRODUCING CYCLOALKYL BROMIDE

(71) Applicant: SUMITOMO CHEMICAL COMPANY, LIMITED, Tokyo (JP)

(72) Inventors: Ryosuke KATO, Osaka-shi (JP); Takahiro KIMURA, Itami-shi (JP); Soichi ITO, Osaka-shi (JP)

(73) Assignee: SUMITOMO CHEMICAL COMPANY, LIMITED, Tokyo (JP)

(21) Appl. No.: 18/562,986

(22) PCT Filed: Apr. 15, 2022
(86) PCT No.: PCT/JP2022/017906

§ 371 Date: Nov. 21, 2023

(30) Foreign Application Priority Data
May 28, 2021 (JP) 2021-089949

(51) Int. Cl. C07C 17/093; C07C 23/04; C07C 23/06

(52) U.S. Cl. CPC C07C 17/093; C07C 23/04; C07C 23/06

(57) ABSTRACT

An object of the present invention is to provide a method for producing a cycloalkyl bromide without using heavy metals and the like. The present invention provides a method for producing a compound represented by Formula (2) wherein R represents an optionally substituted C3-C4 cycloalkyl group, by reacting a compound represented by Formula (1) wherein R represents the same meaning as described above, and M represents an alkali metal, with bromine in presence of a radical initiator or under light irradiation.

### TECHNICAL FIELD

The present invention relates to a method for producing a cycloalkyl bromide.

### BACKGROUND ART

A cycloalkyl bromide is a useful compound which can be converted into various compounds including a cycloalkyl magnesium bromide and a cycloalkyl boronic acid compound which are used as intermediates of active ingredient compounds in agro-pharmaceutical field.

For example, Patent Document 1 and Patent Document 2 describe synthesis examples of pharmaceuticals using cyclopropylmagnesium bromide and cyclopropylboronic acid.

As a method for synthesizing a cycloalkyl bromide, a method using a stoichiometric amount of heavy metal is known. For example, Patent Document 3 and Non-Patent Document 1 are known.

### PRIOR ART DOCUMENTS

### Patent Documents

Patent Document 1: WO 2006/26356
Patent Document 2: WO 2011/042918
Patent Document 3: CN 107915689

### Non-Patent Document

Non-Patent Document 1: Journal of the American Chemical Society, 1951, p. 3176

### SUMMARY OF THE INVENTION

### Problems to be Solved by the Invention

An object of the present invention is to provide a method for producing a cycloalkyl bromide without using heavy metals and the like.

### Solutions to the Problems

As a result of studying a method for producing a cycloalkyl bromide, the present inventors have found that a desired cycloalkyl bromide can be produced without using heavy metals and the like by using a potassium salt of a cycloalkyl carboxylic acid. That is, the present invention is as follows.

[1] A method for producing a compound represented by Formula (2)

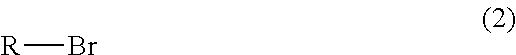

wherein R represents an optionally substituted C3-C4 cycloalkyl group, by reacting a compound represented by Formula (1)

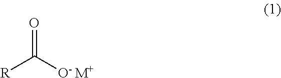

wherein R represents the same meaning as described above, and M represents an alkali metal,
with bromine in presence of a radical initiator or under light irradiation.

[2] The method according to [1], wherein in the compound represented by Formula (1), R is a cyclopropyl group or a cyclobutyl group.

[3] The method according to [1] or [2], wherein in the compound represented by Formula (1), M is potassium or cesium.

[4] The method according to any one of [1] to [3], wherein the radical initiator is an azo compound.

[5] The method according to any one of [1] to [4], which is carried out in presence of a solvent.

[6] The method according to [5], wherein a halogenated hydrocarbon, a nitrile, or an ester is used as the solvent.

[7] The method according to [5] or [6], wherein an aryl chloride, a C1-C3 alkylnitrile, a benzonitrile, or a C1-C6 alkyl acetate ester is used as the solvent.

### Effects of the Invention

According to the present invention, a cycloalkyl bromide can be synthesized without using heavy metals and the like.

### DETAILED DESCRIPTION

A substituent in the present invention will be described.

The expression of “CY-CZ” as used herein represents that the number of carbon atoms is from Y to Z. For example, the expression of “C1-C6” represents that the number of carbon atoms is from 1 to 6.

Examples of the C1-C6 alkyl group include a methyl group, an ethyl group, a propyl group, an isopropyl group, a 1,1-dimethylpropyl group, a 1,2-dimethylpropyl group, a 1-ethylpropyl group, a butyl group, a sec-butyl group, a tert-butyl group, a pentyl group, and a hexyl group.

Examples of the C1-C6 alkoxy group include a methoxy group, an ethoxy group, a propoxy group, an isopropoxy group, a butoxy group, a tert-butoxy group, a pentyloxy group, and a hexyloxy group.

Examples of the C2-C7 alkylcarbonyl group include an acetyl group, a propanoyl group, a butanoyl group, a 2-methylpropanoyl group, a pentanoyl group, a hexanoyl group, and a heptanoyl group.

Examples of the C2-C7 alkoxycarbonyl group include a methoxycarbonyl group, an ethoxycarbonyl group, a propoxycarbonyl group, an isopropyloxycarbonyl group, a butoxycarbonyl group, a pentyloxycarbonyl group, and a hexyloxycarbonyl group.

Examples of the C1-C6 alkylthio group include a methylthio group, an ethylthio group, a propylthio group, an isopropylthio group, a butylthio group, a pentylthio group, and a hexylthio group.

Examples of the C1-C6 alkylsulfinyl group include a methanesulfinyl group, an ethanesulfinyl group, a propanesulfinyl group, a propane-2-ylsulfinyl group, a butanesulfinyl group, a pentanesulfinyl group, and a hexanesulfinyl group.

Examples of the C1-C6 alkylsulfonyl group include a methanesulfonyl group, an ethanesulfonyl group, a propanesulfonyl group, a propane-2-ylsulfonyl group, a butanesulfonyl group, a pentanesulfonyl group, and a hexanesulfonyl group.

Examples of the di(C1-C6 alkyl) aminocarbonyl group include a dimethylaminocarbonyl group, an ethylmethylaminocarbonyl group, a diisopropylcarbonyl group, and a dihexylaminocarbonyl group.

Examples of the (C2-C7 alkylcarbonyl optionally substituted with one or more halogen atoms) (C1-C6 alkyl) amino group include an N-methylacetamino group, an N-methyl-2,2,2-trifluoroacetamino group, and an N-hexylheptanoylamino group.

The halogen atom means a fluorine atom, a chlorine atom, a bromine atom, or an iodine atom.

Examples of the aryl group include a phenyl group, a naphthyl group, an indanyl group, and a tetrahydronaphthyl group.

Examples of the heteroaryl group include a pyrrolyl group, a furyl group, a thienyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a pyridyl group, a pyridazinyl group, a pyrimidinyl group, a pyrazinyl group, a triazinyl group, and a tetrazinyl group.

When the substituent is substituted with two or more halogen atoms or substituents, the halogen atoms or substituents may be the same or different.

Examples of the substituent which may be possessed by the C3-C4 cycloalkyl group represented by R in Formula (1) include one or more substituents selected from Group A.

Group A: a group consisting of C1-C6 alkyl groups optionally substituted with one or more substituents selected from Group B, C3 to C7 cycloalkyl groups optionally substituted with one or more substituents selected from Group C, aryl groups optionally substituted with one or more substituents selected from group D, heteroaryl groups optionally substituted with one or more substituents selected from group D, OR¹, OS(O)ₘR¹, OC(O)R¹, NR¹R², NR¹NR²R³, NR²OR¹, NR²C(O)R¹, NR²NR³C(O)R¹, NR²C(O)OR¹, NR²NR³C(O)OR¹, NR¹C(O)NR²R³, NR²S(O)₂R¹, C(O)R¹, C(O)OR¹, C(O)NR¹R², C(O)NR²S(O)₂R¹, CR²═NOR¹, S(O)ₘR¹, a cyano group, a nitro group, a formyl group, or a halogen atom.

R¹, R², and R³ are the same or different and each represents a C1-C6 alkyl group optionally substituted with one or more substituents selected from Group B, a C3-C7 cycloalkyl group optionally substituted with one or more substituents selected from Group C, or an aryl group optionally substituted with one or more substituents selected from Group D, and
m represents 0, 1, or 2.

Group B: a group consisting of a C3-C7 cycloalkyl group optionally substituted with one or more substituents selected from Group C, a C1-C6 alkoxy group optionally substituted with one or more halogen atoms, a C2-C7 alkylcarbonyl group optionally substituted with one or more halogen atoms, a C2-C7 alkoxycarbonyl group optionally substituted with one or more halogen atoms, a C1-C6 alkylthio group optionally substituted with one or more halogen atoms, a C1-C6 alkylsulfinyl group optionally substituted with one or more halogen atoms, a C1-C6 alkylsulfonyl group optionally substituted with one or more halogen atoms, a di(C1-C6 alkyl)aminocarbonyl group, a (C2-C7 alkylcarbonyl optionally substituted with one or more halogen atoms) (C1-C6 alkyl)amino group, an aryl group optionally substituted with one or more substituents selected from Group E, an aryloxy group optionally substituted with one or more substituents selected from Group E, a cyano group, a nitro group, a formyl group, and a halogen atom.

Group C: a group consisting of a C1-C6 alkyl group optionally substituted with one or more halogen atoms, a C1-C6 alkoxy group optionally substituted with one or more halogen atoms, a C2-C7 alkylcarbonyl group optionally substituted with one or more halogen atoms, a C2-C7 alkoxycarbonyl group optionally substituted with one or more halogen atoms, a C1-C6 alkylthio group optionally substituted with one or more halogen atoms, a C1-C6 alkylsulfinyl group optionally substituted with one or more halogen atoms, a C1-C6 alkylsulfonyl group optionally substituted with one or more halogen atoms, a di(C1-C6 alkyl)aminocarbonyl group, a (C2-C7 alkylcarbonyl optionally substituted with one or more halogen atoms) (C1-C6 alkyl)amino group, an aryl group optionally substituted with one or more substituents selected from Group E, an aryloxy group optionally substituted with one or more substituents selected from Group E, a cyano group, a nitro group, a formyl group, and a halogen atom.

Group D: a group consisting of a C3-C7 cycloalkyl group optionally substituted with one or more substituents selected from Group E, a C1-C6 alkyl group optionally substituted with one or more halogen atoms, a C1-C6 alkoxy group optionally substituted with one or more halogen atoms, a C2-C7 alkyl carbonyl group optionally substituted with one or more halogen atoms, a C2-C7 alkoxycarbonyl group optionally substituted with one or more halogen atoms, a C1-C6 alkylthio group optionally substituted with one or more halogen atoms, a C1-C6 alkylsulfinyl group optionally substituted with one or more halogen atoms, a C1-C6 alkylsulfonyl group optionally substituted with one or more halogen atoms, a di(C1-C6 alkyl)aminocarbonyl group, a (C2-C7 alkylcarbonyl optionally substituted with one or more halogen atoms) (C1-C6 alkyl)amino group, an aryl group optionally substituted with one or more substituents selected from Group E, an aryloxy group optionally substituted with one or more substituents selected from Group E, a cyano group, a nitro group, a formyl group, and a halogen atom.

Group E: a group consisting of a C1-C6 alkyl group optionally substituted with one or more halogen atoms, a C1-C6 alkoxy group optionally substituted with one or more halogen atoms, a C2-C7 alkylcarbonyl group optionally substituted with one or more halogen atoms, a C2-C7 alkoxycarbonyl group optionally substituted with one or more halogen atoms, a C1-C6 alkylthio group optionally substituted with one or more halogen atoms, a C1-C6 alkylsulfinyl group optionally substituted with one or more halogen atoms, a C1-C6 alkylsulfonyl group optionally substituted with one or more halogen atoms, a cyano group, a nitro group, and a halogen atom.

Preferably, examples of the substituent which may be possessed by the C3-C4 cycloalkyl group represented by R include one or more substituents selected from Group F.

Group F: a group consisting of a C1-C6 alkyl group optionally substituted with one or more substituents selected from Group G, a C3-C7 cycloalkyl group optionally substituted with one or more substituents selected from Group H, an aryl group optionally substituted with one or more substituents selected from Group H, OR¹, S(O)ₘR¹, or a halogen atom.

Group G: a group consisting of a C3-C7 cycloalkyl group optionally substituted with one or more halogen atoms, a C1-C6 alkoxy group optionally substituted with one or more halogen atoms, an aryl group, an aryloxy group {the aryl group and the aryloxy group are optionally substituted with one or more substituents selected from the group consisting of a C1-C6 alkyl group optionally substituted with one or more halogen atoms and a halogen atom}, and a halogen atom.

Group H: a group consisting of a C1-C6 alkyl group optionally substituted with one or more halogen atoms, a C3-C7 cycloalkyl group optionally substituted with one or more halogen atoms, a C1-C6 alkoxy group optionally substituted with one or more halogen atoms, an aryl group, an aryloxy group {the aryl group and the aryloxy group are optionally substituted with one or more substituents selected from the group consisting of a C1-C6 alkyl group optionally substituted with one or more halogen atoms and a halogen atom}, and a halogen atom.

More preferably, examples of the substituent which may be possessed by the C3-C4 cycloalkyl group represented by R include a C1-C6 alkyl group optionally substituted with one or more halogen atoms, an aryl group optionally substituted with one or more halogen atoms, a C3-C7 cycloalkyl group optionally substituted with one or more halogen atoms, a C1-C6 alkoxy group optionally substituted with one or more halogen atoms, and a C3-C4 cycloalkyl group optionally substituted with one or more substituents selected from the group consisting of halogen atoms, and R is still more preferably a C3-C4 cycloalkyl group.

Examples of the alkali metal represented by M in Formula (1) include lithium, sodium, potassium, rubidium, and cesium, potassium and cesium are preferable, and potassium is more preferable.

The present reaction will be described.

When the present reaction is carried out in the presence of a solvent, examples of the solvent include halogenated hydrocarbons such as alkyl chloride, aryl chloride, alkyl bromide, and aryl bromide; nitriles such as C1-C3 alkyl nitrile and aromatic nitrile; esters such as C1-C6 alkyl acetate and C1-C6 alkyl benzoate; ethers such as tetrahydrofuran, 2-methyltetrahydrofuran, cyclopentyl methyl ether, diethyl ether, and polyethylene glycol; and carbonic acid esters such as dimethyl carbonate, diethyl carbonate, ethylene carbonate, and propylene carbonate. Examples of the alkyl chloride include dichloromethane and chloroform; examples of the aryl chloride include monochlorobenzene, o-dichlorobenzene, and p-dichlorobenzene; examples of the alkyl bromide include bromomethane; examples of the aryl bromide include bromobenzene; examples of the C1-C3 alkyl nitrile include acetonitrile and propionitrile; examples of the aromatic nitrile include benzonitrile; examples of the C1-C6 alkyl acetate include ethyl acetate, isopropyl acetate, and butyl acetate; and examples of the C1-C6 benzoate alkyl ester include methyl benzoate, ethyl benzoate, and butyl benzoate.

Preferred solvents are halogenated hydrocarbons, nitriles, and esters, more preferred solvents are aryl chloride, C1-C3 alkylnitrile, benzonitrile, and C1-C6 acetic acid alkyl ester, and still more preferred solvents are monochlorobenzene, acetonitrile, benzonitrile, and butyl acetate. The solvent may be only one kind, or a plurality of solvents may be used in combination.

An amount of the solvent used is 0.1 to 100 parts by weight, preferably 0.5 to 10 parts by weight, based on 1 part by weight of the compound represented by Formula (1) (hereinafter, referred to as the compound (1)).

An amount of bromine used is usually a proportion of 0.5 to 10 mol, and preferably a proportion of 0.8 to 2.0 mol, based on 1 mol of the compound (1).

An amount of the radical initiator used is usually a proportion of 0.001 to 1.0 mol, and preferably a proportion of 0.01 to 0.1 mol, based on 1 mol of the compound (1).

Examples of the radical initiator include azo compounds such as azobisisobutyronitrile (AIBN), 2,2′-azobis(4-methoxy-2,4-dimethylvaleronitrile (V-70), 2,2′-azobis [2-(2-imidazoline-2-yl)propane] dihydrochloride (VA-044), and 2,2′-azobis (2-methylpropionamidine) dihydrochloride (V-50); and peroxides such as tert-butyl hydroperoxide and benzoyl peroxide, an azo compound is preferable, and azobisisobutyronitrile is more preferable.

When the present reaction is carried out under light irradiation, examples of the light source include a high-pressure mercury lamp, a low-pressure mercury lamp, and an incandescent lamp, and a high-pressure mercury lamp is preferable.

A radical initiator and light irradiation may be used in combination.

The reaction temperature is usually in a range of 0° C. to 200° C., preferably 30° C. to 100° C., and more preferably 60° C. to 80° C.

The reaction time is usually 0.1 to 100 hours, and preferably 0.5 to 10 hours.

Examples of a method of mixing raw materials in the present reaction include a batch method in which all the raw materials are added to one container, and a flow method using a flow reactor.

Examples of the batch-type mixing method include a method in which a radical initiator is added to a mixed solution of the compound (1) and a solvent and then bromine is added dropwise, and a method in which a mixed solution of the compound (1) and a solvent and bromine are simultaneously added dropwise to a mixed solution of the solvent and a radical initiator. If necessary, the mixed solution of the compound (1) and the solvent may be treated by a moisture removal operation by concentration or addition of a dehydrating agent. When the radical initiator is carried out by light irradiation, there is a method in which bromine is added dropwise to the mixed solution of the compound (1) and the solvent while light irradiation is carried out. In each case, bromine may be added as it is, or may be added after being diluted with a solvent. The time for dropwise addition of bromine is usually 0.1 to 100 hours, and preferably 0.1 to 24 hours. In the flow method, there is a method of passing the compound (1), a mixed solution of the radical initiator and the solvent, and bromine through the flow reactor. When the radical initiator is carried out by light irradiation, there is a method of passing the mixed solution of the compound (1) and the solvent and bromine through a flow reactor irradiated with light.

After completion of the reaction, post-treatment operations, such as distillation of a reaction mixture; and after mixing of the reaction mixture with neutral or weakly basic water, extraction with an organic solvent, and drying or distillation of the resulting organic layer, are performed, whereby a compound (2) can be isolated.

The isolated compound (2) can be further purified by a method such as distillation, recrystallization, or chromatography.

As the compound (1), a commercially available compound may be used, or a compound synthesized by a known method may be used. As a method of synthesizing the compound (1), for example, the compound (1) can be obtained by neutralization reaction of a corresponding carboxylic acid, a base such as an alkali metal hydroxide such as sodium hydroxide, potassium hydroxide, or cesium hydroxide; an alkali metal alkoxide such as sodium methoxide, sodium ethoxide, sodium t-butoxide, potassium methoxide, potassium ethoxide, or potassium tert-butoxide; an alkali metal carbonate such as sodium carbonate, sodium bicarbonate, potassium carbonate, potassium bicarbonate, or cesium carbonate; an alkali metal phosphate such as trisodium phosphate, disodium hydrogen phosphate, sodium dihydrogen phosphate, tripotassium phosphate, dipotassium hydrogen phosphate, or potassium dihydrogen phosphate; an alkali metal hydride such as sodium hydride or potassium hydride; an alkali metal amide such as sodium amide and potassium amide; sodium hexamethyldisilazide, potassium hexamethyldisilazide; and a carboxylic acid in which M is a hydrogen atom in Formula (1).

Since the present reaction is affected by moisture, a moisture amount of a reaction system is preferably 1.0% by weight or less, more preferably 0.2% by weight or less with respect to the compound (1). Thus, it is preferable to remove moisture from the compound (1). A moisture concentration of the compound (1) is preferably 1.0% by weight or less, and more preferably 0.2% by weight or less. A method of removing moisture from the compound (1) is, for example, a method of allowing the compound (1) to act on a dehydrating agent such as anhydrous sodium sulfate, anhydrous magnesium sulfate, or molecular sieves; or a method of removing moisture by concentration. For example, performing azeotropic dehydration after a neutralization reaction can remove moisture from (1) synthesized by reacting a corresponding carboxylic acid and an alkali metal hydroxide. When an alkali metal alkoxide is used, the compound (1) suitable for the reaction can be obtained by concentrating alcohol generated by the neutralization reaction. When an alkali metal carbonate, an alkali metal phosphate, an alkali metal hydride, or an alkali metal amide is used as a base, the solution after the neutralization reaction can be used as it is in the present reaction without isolating the compound (1).

### EXAMPLES

Hereinafter, the present invention will be described based on examples. The contents of the compound (1) and the compound (2) were each determined by an internal standard method by gas chromatography using a commercially available product as a sample.

### Reference Example 1

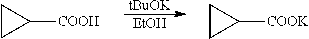

Under a nitrogen atmosphere, 26.07 g of tert-butoxypotassium was added to a mixture of 20 g of cyclopropanecarboxylic acid and 200 g of ethanol at 20° ° C., and the mixture was stirred at 50ºC for 1 hour. The resulting mixture was concentrated to obtain 27.26 g of potassium cyclopropanecarboxylate having a moisture concentration of 0.0064% by weight.

### Example 1

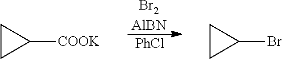

Under a nitrogen atmosphere, a mixture of 9.65 g of bromine and 15 g of monochlorobenzene was added dropwise to a mixture of 7.5 g of potassium cyclopropanecarboxylate which was obtained in Reference Example 1, 0.496 g of AIBN, and 22.5 g of monochlorobenzene at 70° ° C. over 2 hours. The resulting mixture was analyzed by a gas chromatography internal standard method, and it was confirmed that 4.90 g of cyclopropyl bromide was contained. (Yield 67.6%)

### Example 2

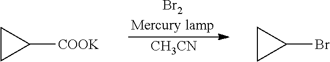

Under a nitrogen atmosphere, a mixture of 9.65 g of bromine and 30 g of acetonitrile was added dropwise to a mixture of 7.5 g of potassium cyclopropanecarboxylate which was obtained in Reference Example 1 and 45 g of acetonitrile over 2 hours, under light (light source: 400 W high-pressure mercury lamp) irradiation at 70° C. After stirring for 2 hours, the resulting mixture was analyzed by the gas chromatography internal standard method to confirm that 4.82 g of cyclopropyl bromide was contained. (Yield 65.9%)

### Example 3

Cyclopropyl bromide was obtained in a yield of 54.5% in the same manner as in Example 1 except that acetonitrile was used as the solvent instead of monochlorobenzene.

### Example 4

Cyclopropyl bromide was obtained in a yield of 52.1% in the same manner as in Example 1 except that benzonitrile was used as the solvent instead of monochlorobenzene.

### Example 5

Cyclopropyl bromide was obtained in a yield of 50.3% in the same manner as in Example 1 except that butyl acetate was used as the solvent instead of monochlorobenzene.

### Reference Example 2

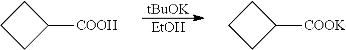

Under a nitrogen atmosphere, 2.24 g of tert-butoxypotassium was added to a mixture of 2 g of cyclobutanecarboxylic acid and 20 g of ethanol at 20° C., and the mixture was stirred at 50° ° C. for 1 hour. The resulting mixture was concentrated to obtain 2.54 g of potassium cyclobutanecarboxylate having a moisture concentration of 0.20% by weight.

### Example 6

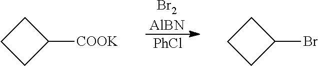

Under a nitrogen atmosphere, a mixture of 0.58 g of bromine and 2.0 g of monochlorobenzene was added dropwise to a mixture of 0.5 g of potassium cyclobutanecarboxylate which was obtained in Reference Example 2, 0.03 g of AIBN, and 3.0 g of monochlorobenzene at 70° ° C. over 2 hours. The resulting mixture was analyzed by the gas chromatography internal standard method, and it was confirmed that 0.36 g of cyclobutyl bromide was contained. (Yield 75.0%)

### Example 7

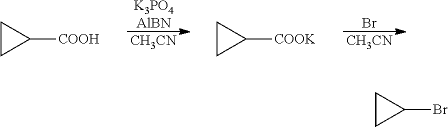

Under a nitrogen atmosphere, 5.0 g of cyclopropanecarboxylic acid, 0.477 g of AIBN, 6.16 g of tripotassium phosphate, and 30 g of acetonitrile were mixed at room temperature to obtain a mixture containing potassium cyclopropanecarboxylate. Then, a mixture of 9.28 g of bromine and 20 g of acetonitrile was added dropwise to the mixture at 70° C. over 2 hours. After stirring for 3 hours, the resulting mixture was analyzed by the gas chromatography internal standard method to confirm that 3.82 g of cyclopropyl bromide was contained. (Yield 54.3%)

### Example 8

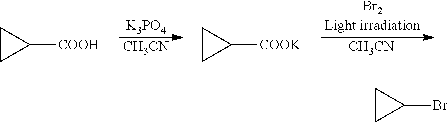

Under a nitrogen atmosphere, 5.0 g of cyclopropanecarboxylic acid, 6.16 g of tripotassium phosphate, and 30 g of acetonitrile were mixed at room temperature to obtain a mixture containing potassium cyclopropanecarboxylate. Then, a mixture of 9.28 g of bromine and 20 g of acetonitrile was added dropwise to the mixture at 70° C. under light (light source: 400 W high-pressure mercury lamp) irradiation over 2 hours. After stirring for 4 hours, the resulting solution was analyzed by the gas chromatography internal standard method to confirm that 4.61 of cyclopropyl bromide was contained. (Yield 65.7%)

### Reference Example 3

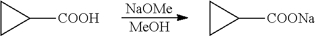

Under a nitrogen atmosphere, 11.2 g of sodium methoxide was added to a mixture of 5 g of cyclopropanecarboxylic acid and 50 g of methanol at 20° C., and the mixture was stirred at 50° ° C. for 1 hour. The resulting mixture was concentrated to obtain 6.97 g of sodium cyclopropane carboxylate having a moisture concentration of 0.29% by weight.

### Example 9

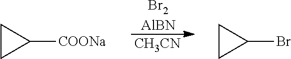

Under a nitrogen atmosphere, a mixture of 0.74 g of bromine and 2.0 g of acetonitrile was added dropwise to a mixture of 0.5 g of sodium cyclopropanecarboxylate, 0.038 g of AIBN, and 3.0 g of acetonitrile at 70° ° C. over 2 hours. After stirring at the same temperature for 4 hours, the resulting mixture was analyzed by the gas chromatography internal standard method, and it was confirmed that 0.14 g of cyclopropyl bromide was contained. (Yield 24.1%)

### Reference Example 4

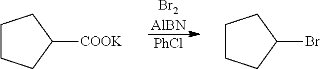

Under a nitrogen atmosphere, a mixture of 0.52 g of bromine and 2.0 g of monochlorobenzene was added dropwise to a mixture of 0.5 g of potassium cyclopentanecarboxylate, 0.03 g of AIBN, and 3.0 g of monochlorobenzene at 70° C. over 2 hours. The resulting mixture was analyzed by the gas chromatography internal standard method, and it was confirmed that 0.1 g of cyclopentyl bromide was contained. (Yield 20.7%)

### Example 10

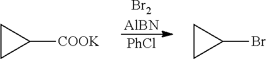

Cyclopropyl bromide was obtained in a yield of 31.3% in the same manner as in Example 1 except that potassium cyclopropanecarboxylate having a moisture content of 0.99% by weight was used.

### Example 11

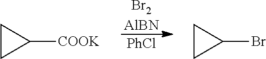

Cyclopropyl bromide was obtained in a yield of 56.3% in the same manner as in Example 1 except that potassium cyclopropyl carboxylate having a moisture concentration of 0.010% by weight was used.

### Reference Example 5

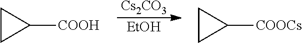

Under a nitrogen atmosphere, 18.9 g of cesium carbonate was added to a mixture of 10 g of cyclopropanecarboxylic acid and 50 g of ethanol at 20° C., and the mixture was stirred at 50° C. for 1 hour. The solvent was distilled off from the resulting mixture under reduced pressure. The resulting cesium cyclopropanecarboxylate was refluxed and dehydrated in chlorobenzene, and then the solvent was distilled off under reduced pressure to obtain 23.88 g of cesium cyclopropanecarboxylate having a moisture concentration of 0.009% by weight.

### Example 12

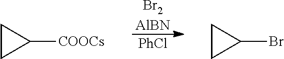

Under a nitrogen atmosphere, a mixture of 7.33 g of bromine and 10 g of chlorobenzene was added dropwise to a mixture of 10 g of cesium cyclopropanecarboxylate, 0.377 g of AIBN, and 40 g of chlorobenzene at 70° ° C. over 2 hours. After stirring at the same temperature for 4 hours, the resulting mixture was analyzed by the gas chromatography internal standard method, and it was confirmed that 3.77 g of cyclopropyl bromide was contained. (Yield 68.0%)

### [GC Analysis Conditions]

Column: DB-WAX (0.25 μm×0.25 mmΦ×30 m)

Inlet temperature: 250° ° C.

Detector: FID, 250° C.

Control mode: linear velocity pressure: 188.2 kPa, linear velocity: 55.7 cm/sec

Carrier gas: helium

Flow rate: 3.23 mL/min

Injection amount: 1.0 μL (split ratio 50:1)

Oven temperature: 40° ° C. (20 min)→20° C./min→250° C. (20 min)

### INDUSTRIAL APPLICABILITY

According to the present invention, a cycloalkyl bromide, which is a compound useful as a raw material for producing an active ingredient compound in agro-pharmaceutical field, can be synthesized without using heavy metals and the like.

1: A method for producing a compound of Formula (2) 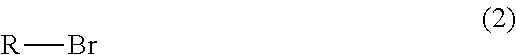
wherein R is an optionally substituted C3-C4 cycloalkyl group, comprising:
reacting a compound of Formula (1) with bromine in presence of a radical initiator or under light irradiation, 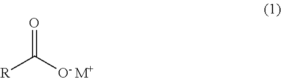
wherein in the Formula (1), R is an optionally substituted C3-C4 cycloalkyl group, and M is an alkali metal.

2: The method according to claim 1, wherein in the Formula (1), R is a cyclopropyl group or a cyclobutyl group.

3: The method according to claim 1, wherein in the Formula (1), M is potassium or cesium.

4: The method according to claim 1, wherein the reacting is carried out in the presence of the radical initiator comprising an azo compound.

5: The method according to claim 1, wherein the reacting is carried out in presence of a solvent.

6: The method according to claim 5, wherein the solvent comprises a halogenated hydrocarbon, a nitrile, or an ester.

7: The method according to claim 5, wherein the solvent comprises an aryl chloride, a C1-C3 alkylnitrile, a benzonitrile, or a C1-C6 alkyl acetate ester.

8: The method according to claim 2, wherein in the Formula (1), M is potassium or cesium.

9: The method according to claim 2, wherein the reacting is carried out in the presence of the radical initiator comprising an azo compound.

10: The method according to claim 3, wherein the reacting is carried out in the presence of the radical initiator comprising an azo compound.

11: The method according to claim 2, wherein the reacting is carried out in presence of a solvent.

12: The method according to claim 3, wherein the reacting is carried out in presence of a solvent.

13: The method according to claim 4, wherein the reacting is carried out in presence of a solvent.

14: The method according to claim 11, wherein the solvent comprises a halogenated hydrocarbon, a nitrile, or an ester.

15: The method according to claim 12, wherein the solvent comprises a halogenated hydrocarbon, a nitrile, or an ester.

16: The method according to claim 13, wherein the solvent comprises a halogenated hydrocarbon, a nitrile, or an ester.

17: The method according to claim 11, wherein the solvent comprises an aryl chloride, a C1-C3 alkylnitrile, a benzonitrile, or a C1-C6 alkyl acetate ester.

18: The method according to claim 12, wherein the solvent comprises an aryl chloride, a C1-C3 alkylnitrile, a benzonitrile, or a C1-C6 alkyl acetate ester.

19: The method according to claim 13, wherein the solvent comprises an aryl chloride, a C1-C3 alkylnitrile, a benzonitrile, or a C1-C6 alkyl acetate ester.

20: The method according to claim 4, wherein the azo compound comprises at least one selected from the group consisting of azobisisobutyronitrile, 2,2′-azobis(4-methoxy-2,4-dimethylvaleronitrile, 2,2′-azobis [2-(2-imidazoline-2-yl)propane] dihydrochloride, and 2,2′-azobis (2-methylpropionamidine) dihydrochloride.
